(19) United States
(12) Patent Application Publication
(10) Pub. No.: US 2013/0237842 A1
(43) Pub. Date: Sep. 12, 2013

(54) DETERMINING CONDITION OF TISSUE USING SPECTRAL ANALYSIS

(71) Applicant: Douglas M. Hawkins, Edina, MN (US)

(72) Inventor: Douglas M. Hawkins, Edina, MN (US)

(73) Assignee: SpectraScience, Inc., San Diego, CA (US)

(21) Appl. No.: 13/413,607

(22) Filed: Mar. 6, 2012

(51) Int. Cl. A61B 6/00

(52) U.S. Cl. 600/476

(57) ABSTRACT

A system for determining a condition of a tissue of a patient body is described. The tissue is illuminated with an illumination wavelength by a light source. In response to the illumination, the tissue emits light. This emitted light is received at a detector that includes multiple diode sensors. The diode sensors detect intensities of associated wavelengths of the emitted light. A spectral analysis is performed with the detected intensities. The spectral analysis includes initial coefficients. A composite function associated with the initial coefficients is minimized so as to determine wavelength coefficients. The wavelength coefficients are used to compute a score. Based on the score, the condition of the tissue is determined. Related methods, techniques, apparatus, and articles are also described.

### TECHNICAL FIELD

The current subject matter relates to the diagnosis of a disease. More particularly, the current subject matter relates to an in-vivo diagnosis performed by optical methods, wherein a penalty function is used to improve accuracy and reliability of the diagnosis.

### BACKGROUND

Determining the condition of a tissue has usually been performed by a combination of electromagnetic techniques followed by biopsies when tissue anomalies such as polyps or lumps are identified. Historically, most biopsies have been performed with limited objective knowledge of the likelihood of the anomaly being diseased or normal. Thus, it may be advantageous to improve the likelihood of knowing the tissue state before performing a biopsy so as to reduce the number of biopsies performed.

### SUMMARY

A system for determining a condition of a tissue of a patient body is described. The tissue is illuminated with an illumination wavelength by a light source. In response to the illumination, the tissue emits light. This emitted light is received at a detector that includes multiple diode sensors. The diode sensors detect intensities of associated wavelengths of the emitted light. A spectral analysis is performed with the detected intensities. The spectral analysis includes initial coefficients. A composite function associated with the initial coefficients is minimized so as to determine wavelength coefficients. The wavelength coefficients are used to compute a score. Based on the score, the condition of the tissue is determined. Related methods, techniques, apparatus, and articles are also described.

In one aspect, a tissue of a body is excited with an excitation wavelength. In response to the illumination, light is received from the tissue. Intensities associated with wavelengths of the received light are detected. Using a plurality of wavelength dependent coefficients determined using a composite function that includes a second function applied to minimize differences between neighboring coefficients, a score is computed. The score is characterized by a weighted function of the intensities. Based on the score, an output characterizing a condition of the tissue is generated.

In one variation, the composite function comprises a first function and the second function. The plurality of wavelength dependent coefficients are determined by minimizing the composite function.

In another variation, a plurality of wavelength dependent coefficients are received.

In yet another variation, the first function is a least squares regression function applied to train data.

In one variation, the second function includes a sum of squared differences between neighboring coefficients of the plurality of coefficients.

In another variation, the excitation wavelength is about 337 nanometers, and the spectral data is associated with a spectral curve disposed between 350 nm and 600 nm.

In one variation, the condition of the tissue characterizes whether the tissue is diseased.

In another variation, the intensities are detected using a plurality of diodes, each diode being sensitive to a respective band of wavelengths, each detected intensity of the intensities being based on an output of one or more diodes of the plurality of diodes.

In another variation, the intensities are normalized such that the intensity values are dimensionless.

In one aspect, a system is described that comprises at least one programmable processor, and a non-transitory machine-readable medium. The machine-readable medium stores instructions that, when executed by the at least one processor, cause the at least one programmable processor to perform operations comprising: receiving data regarding wavelengths of light emitted from a tissue; determining, based on the wavelengths in the received data and using a composite function comprising a mathematical sum of a first function and a second function, coefficients of spectral analysis data, the second function minimizing differences between neighboring coefficients; determining, based on the wavelengths in the received data and using a composite function comprising a mathematical sum of a first function and a second function, coefficients of spectral analysis data, the second function minimizing differences between neighboring coefficients; and providing the coefficients, the coefficients being used to generate one or more scores used to generate an output characterizing a condition of the tissue.

In one variation, the coefficients are determined by minimizing the composite function. The first function characterizes a least squares regression analysis performed on spectral data associated with a plurality of individuals, the least squares regression analysis being associated with a plurality of initial coefficients. The second function characterizes a sum of squared differences between each neighboring coefficients of the plurality of initial coefficients.

In another variation, an average absolution value of a difference between consecutive coefficients is less than two percent of a range of the plurality of coefficients.

In another aspect, a system is described that comprises at least one illumination source, at least one detector, and a computational module. The at least one illumination source is configured to illuminate a tissue with an excitation wavelength. The at least one detector is configured to perform operations comprising: receiving, in response to the illumination, light emitted from the tissue; and detecting intensities corresponding to wavelengths of the emitted light. The computational module comprises a non-transitory machine-readable medium storing instructions that, when executed by at least one programmable processor, cause the at least one programmable processor to perform operations comprising: computing, using a plurality of wavelength dependent coefficients determined by minimizing a sum of a first function and a second function, the second function minimizing a difference between neighboring coefficients, a score that is characterized by a weighted function of the intensities; and generating, based on the score, an output characterizing a condition of the tissue.

In one variation, the computational module further performs operations comprising: receiving the plurality of wavelength dependent coefficients.

In another variation, the first function characterizes a least squares regression analysis performed on spectral data associated with a plurality of individuals.

In one variation, the least squares regression analysis is associated with a plurality of coefficients, and wherein the second function includes a sum of terms, each term being proportional to a squared difference between a corresponding coefficient of the plurality of coefficients and an average of at least two coefficients that are nearest neighbors with the corresponding coefficient.

the least squares regression analysis is associated with a plurality of coefficients, and wherein the second function includes a sum of terms, each term being proportional to a squared difference between two consecutive coefficients.

In another variation, the least squares regression analysis is associated with a plurality of coefficients, and wherein the second function includes a sum of terms, each term being proportional to a squared difference between a corresponding coefficient of the plurality of coefficients and an average of at least two coefficients that are nearest neighbors with the corresponding coefficient.

In one variation, the excitation wavelength is 337 nanometers, and the spectral data is associated with a spectral curve disposed between 350 nm and 600 nm.

Articles are also described that comprise a tangibly embodied machine-readable medium operable to cause one or more machines (e.g., computers, etc.) to result in operations described herein. Similarly, computer systems are also described that may include a processor and a memory coupled to the processor. The memory may include one or more programs that cause the processor to perform one or more of the operations described herein.

The subject matter described herein provides many advantages. For example, the system that determines condition of the tissue can be used for any patient, as opposed to conventional spectral curves that may vary from patient to patient. Further, the analysis performed using information extracted stays consistent when different apparatuses are used. That is, there is no variability in results when different apparatuses are used. Furthermore, the described systems are easily compatible with new patient data. Thus, the described techniques stay consistent with variations in patient, system and measurement of data.

The details of one or more variations of the subject matter described herein are set forth in the accompanying drawings and the description below. Other features and advantages of the subject matter described herein will be apparent from the description and drawings, and from the claims.

### BRIEF DESCRIPTION OF THE DRAWINGS

FIG. 1 illustrates exemplary spectral reflectance curves for normal and diseased (adenoma) tissue;

FIG. 2 illustrates a block diagram representation of an exemplary system for determining the condition of tissue;

FIG. 3 illustrates a flow chart representation of an exemplary method in accordance with some implementations of the current subject matter;

FIG. 4 illustrates an exemplary graph of a normalized intensity xⱼ versus a wavelength range number j.

FIG. 5 illustrates an exemplary graph of weighting coefficients bⱼ versus a wavelength range number j that are determined using a composite function in accordance with some implementations of the current subject matter;

FIG. 6 illustrates an exemplary graph of unsmoothed weighting coefficients versus a wavelength range number j; and

FIG. 7 illustrates an exemplary graph of weighting coefficients bⱼ versus a wavelength range number j that are determined using a composite function in accordance with some implementations of the current subject matter.

### DETAILED DESCRIPTION

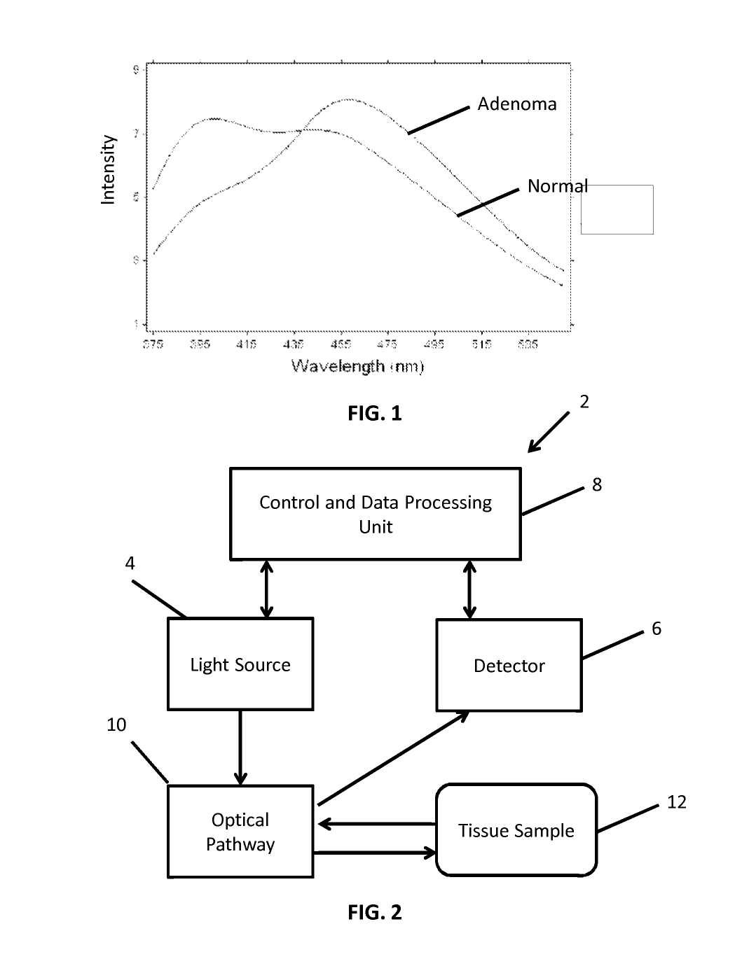

FIG. 1 is an exemplary set of curves illustrating spectral responses of tissues that can be normal or have adenoma. These curves can depict the intensity of light emitted from tissue versus wavelength. The shape of the curve can be indicative of the condition (for example, diseased or not-diseased) of the tissue. Some implementations described herein obviate a possible patient-to-patient variation of some spectral curves.

FIG. 2 illustrates a block diagram of an exemplary system 2 for determining the condition of tissue in accordance with some implementations of the current subject matter. System 2 can include a light source 4 and detector 6 under control of a control and data processing system 8. System 2 can also include an optical pathway 10 that can be configured to direct excitation light from light source 4 to a tissue sample 12 and to direct emitted light from tissue sample 12 to detector 6.

Light source 4 can be configured to generate a wavelength of light that can excite tissue 12. In one implementation, light source 4 can generate a light having a wavelength of 337 nm. In another implementation, light source 4 generates light can have a wavelength of 405 nm. In yet another implementation, light source 4 can emit a plurality of different wavelengths.

In response to receiving excitation light from light source 4, the tissue 12 can emit light having a spectral distribution with a range of wavelengths. In an exemplary implementation, tissue 12 can emit a continuous or nearly continuous spectrum of wavelengths. FIG. 1 illustrates exemplary emission spectra for normal and adenoma tissue showing intensity versus wavelength for light emitted by tissue, which has been excited by a 337 nm light source. As illustrated, the shape of the curve can be different for the two different tissue conditions.

Detector 6 can be configured to receive the emitted light from tissue 12 and to generate a signal that can be indicative of intensities corresponding to wavelengths along a spectral curve, such as one of the spectral curves illustrated in FIG. 1. In one implementation, detector 6 can include a plurality of sensors, each of which can be tuned to a particular wavelength. In a further exemplary implementation, detector 6 can include 1024 sensors, each of which can be sensitive to a narrow wavelength distribution. Detector 6 can also include registers (or other information storage devices) that can contain calibration information that can characterize the sensors in terms of specific wavelength and sensitivity. This calibration information can be used by detector 6 and/or unit 8 to assign a specific wavelength to each sensor for purposes of analysis. The calibration information can also be used to calibrate the signal strength from each sensor so that the relative intensity versus wavelength can be properly ascertained by unit 8. In one implementation, the detector 6 can include a computer including at least one programmable data processor and a non-transitory machine-readable medium storing instructions that, when executed by the at least one processor, cause the at least one programmable processor to perform one or more associated operations.

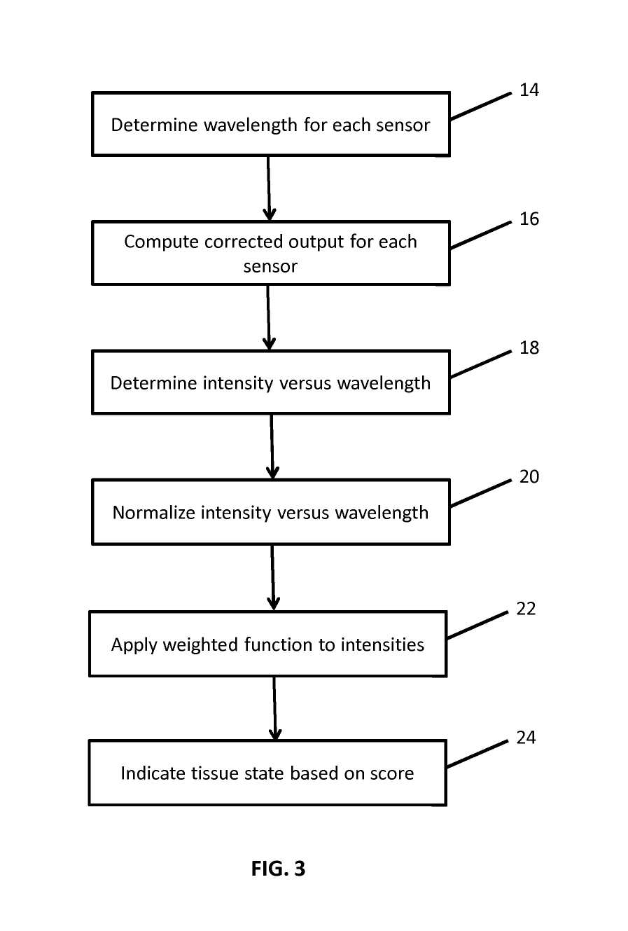

Control and data processing unit 8 can be configured to process the signal indicative of intensities for the wavelengths received by detector 6 so as to indicate the condition of the tissue sample 12. The control and data processing unit 8 can be a computer including at least one programmable data processor and a non-transitory machine-readable medium storing instructions that, when executed by the at least one programmable data processor, cause the at least one programmable processor to perform one or more associated operations. An exemplary implementation of this processing is described in more detail with respect to FIG. 3.

Optical pathway 10 can include a single fiber optic pathway for transmitting light to and from tissue sample 12. Alternatively, optical pathway 10 can include separate optical paths for transmitting light from light source 4 to tissue sample 12 and for transmitting light from tissue sample 12 to detector 6. For receiving light from tissue sample 12, optical pathway can include “on angle” and/or “off angle” collectors depending upon whether coaxially directed emissions, off-axis emissions, isotropic directed emissions, or scattered light emissions are being collected from tissue sample 12. This can be dependent upon the nature of light source 12 which can be a single wavelength light source or a number of different light sources. Additionally, this can be dependent upon the type of tissue that is being observed as well. In an exemplary implementation, the tissue being analyzed can include colon polyps.

FIG. 3 illustrates an exemplary process by which tissue 12 can be illuminated and analyzed in order to receive information indicative of a likely condition of tissue 12. This process can be described for an exemplary detector having 1024 diode sensors, each of which are sensitive to a particular wavelength.

According to step 14, system 2 can determine or assign an integer wavelength for each sensor in detector 6. Step 14 can have two sub-steps. A first sub-step can include the step of determining the wavelength of each sensor using stored calibration information from the sensor manufacturer. A second sub-step can include applying a integer fit “wavelength bucket” to fit each sensor to an integer value in nanometers. In an exemplary implementation, an emitted spectrum from 375 nanometer to 550 nanometers can be used, thereby defining 176 buckets, each of which have a width of one nanometer. The sensors corresponding to each wavelength bucket in this spectral range can therefore be identified and known by system 2. This is one specific example, and other possibilities can exist. For example, the sensors can be fit to smaller increments, such as wavelength buckets that can have a width of 0.75 nanometers, 0.50 nanometers, 0.25 nanometers, or any other selected range of wavelengths along a spectrum. Moreover, other spectral ranges can be utilized.

Each wavelength bucket can be provided with a wavelength number j. The number j can vary from j=1 to j=N with an increase in j corresponding to an increase in wavelength. Each wavelength number j can represent an interval range of wavelengths that can be a portion of the overall range represented by the series from j=1 to j=N. Each sensor can be sensitive to a narrow wavelength range that can correspond to one such wavelength number j.

In this exemplary implementation, N=176. The wavelength number j=1 can correspond to 375 nanometers, wavelength number j=2 can correspond to 376 nanometers, wavelength number j=3 can correspond to 377 nanometers, and so on in one nanometer steps and up to j=176 corresponding to 550 nanometers.

In an alternative implementation, j=1 can correspond to the longest wavelength, and the number j=N can correspond to the shortest wavelength, with each increment of j corresponding to a decrease in wavelength. The wavelength number j can be used to “bucket” one or more sensors of detector 6 for computational purposes. Wavelength and wavelength number can be used interchangeably to indicate a position and wavelength along a spectral curve.

According to step 16, system 2 can compute a corrected output for each sensor (for an actual measurement from tissue sample 12) during a measurement. For each measurement, a background signal and a light source off signal can be subtracted from the signal from the measurement. The background signal can be a signal generated by the sensor in complete darkness. The light source off signal can be the signal that the sensor can generate based upon background light coming from the tissue with the light source 4 turned off. By subtracting the background signal and the light source off signal from each measurement signal with light source on, the signal that is indicative of the light emitted from tissue 12 can be received in response to excitation by light source 4. In one implementation, the process of obtaining the corrected output can be repeated 5 times for each of the 1024 sensors. This is referred to herein as 5 “frames,” wherein each frame can include a single measurement for each of 1024 sensors.

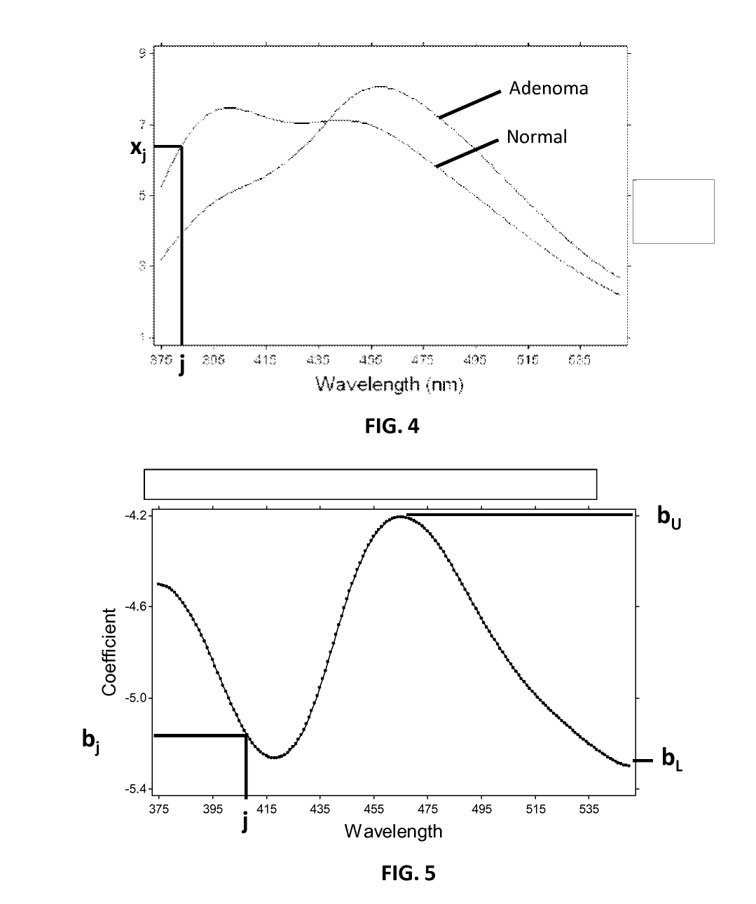

According to step 18, intensity versus wavelength data can be determined from the data generated in step 16. For each wavelength bucket, outputs for each sensor fitting into that bucket can be averaged. Then, the median value for the five frames can be selected. The output from step 18 can be a set of intensities for each set of wavelengths. In an exemplary implementation, there can be 176 intensity values that can correspond to 176 buckets that roughly define a curve, as illustrated in FIG. 4 (shown after normalization). Thus, there can be a series of intensities Iⱼ that can correspond to a series of wavelengths Wⱼ as the output of step 18.

According to step 20, the intensity data can be normalized. In one implementation, a “normalizer” can be computed as the sum of all the intensities over a spectral wavelength range under consideration divided by a certain number, such as the number of buckets N, a number proportional to the number of buckets N, or a constant. Each individual intensity Iⱼ can then be divided by the normalizer to obtain dimensionless intensity value xⱼ. The values xⱼ can form a series of numbers from j=1 to j=N which can characterize the shape of the curve over a spectral range of wavelengths. FIG. 4 illustrates exemplary normal and adenoma shapes defined by the series xⱼ.

According to step 22, a weighting function can be applied to the series xⱼ in order to compute a “score” which can be indicative of the state of the tissue. In an exemplary implementation, there can be a series of coefficients bⱼ, each of which can correspond to one of the series xⱼ according to the number j. In this implementation, the score can be the sum Σbⱼxⱼ for j=1 to j=N. In one implementation, the sum can be calculated for values of j from j=1 to j=176 (all of the intensity values over the wavelength range from 375 to 550 nanometers).

According to step 24, the tissue state can be indicated based upon the computed score. In an exemplary implementation, a diseased curve such as the adenoma curve of FIG. 4 can result in a value of 1 whilst the normal curve results in a value of 0.

The coefficients bⱼ can be defined by applying a composite function to training data that can be based upon observed clinical conditions. The training data can include spectral data from normal and diseased tissue. The spectral data can be used to generate the intensity values xⱼ. Applying the composite function can provide the coefficients bⱼ. A method of applying such a composite function is discussed below. The coefficients bⱼ can then be used to determine whether or not tissue is diseased or normal for new patients using a method that can be similar to that discussed with respect to FIG. 3.

One aspect of this implementation is the reliability and accuracy with which the coefficients bⱼ enable the method of FIG. 3 to more accurately and more reliably predict the condition of new tissue samples for new patients. This is a result of a composite function that is discussed below.

FIG. 5 illustrates an exemplary plot of bⱼ versus j, as j varies from 1 to N. As noted above, each value of j can correspond to a wavelength “bucket range” and values of j can generally increase from the lower end of the wavelength range to the upper end of the wavelength range. One characteristic of the current subject matter is that the bⱼ values vary smoothly with j as illustrated in FIG. 5. In an exemplary implementation, the graphs of the coefficients bⱼ can have a lower bound b_{L} and an upper bound b_{U} over the wavelength range (or j).

The absolute value of a difference between each value bⱼ and its neighboring coefficients bⱼ₋₁ and bⱼ₊₁ can be defined. In the exemplary implementation of FIG. 5, the average value of this absolute value of the difference, which equals average |bⱼ−bⱼ₋₁| over the wavelength range, can be much less than the absolute value of the overall range of the curve |b_{L}−b_{U}|. Thus, average |bⱼ−bⱼ₋₁|<F×|b_{L}−b_{U}| averaged over j=1 to N wherein N can span the wavelength intervals for which the sum can be computed in which F is a fraction less than 0.05 or 5%. In one implementation, F can be less than 0.04 or 4%. In another implementation, F can be less than 0.03 or 3%. In yet another implementation, F can be less than 0.02 or 2%.

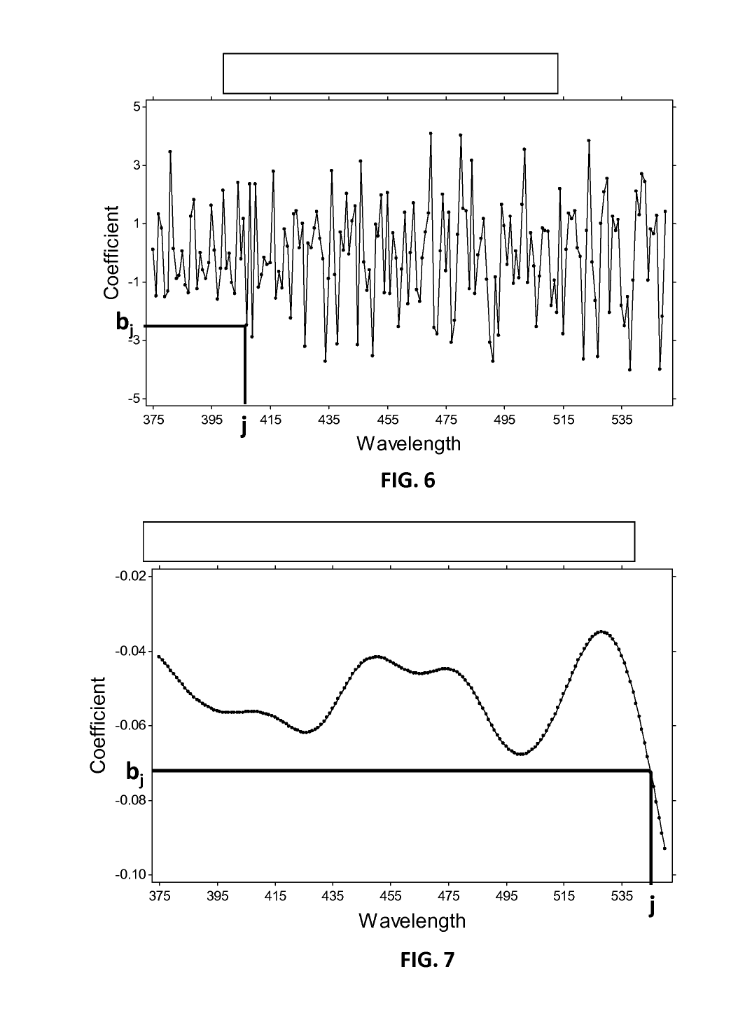

FIG. 6 illustrates values of bⱼ that can be computed using a least squares regression method that can utilize training data. Training data can be spectral data xᵢⱼ that can be obtained from tissue having known conditions yᵢ. The coefficients can be found by minimizing a function such as the following:

Σᵢ₌₁ⁿ[yᵢ−b₀−Σ_{j≦1}^{p}bⱼxᵢⱼ]²

In this equation: n=the number of tissue samples having a known condition that are studied and the outer sum is taken over all n tissue samples; yᵢ is the output as a function of tissue condition; in one implementation yᵢ=0 corresponds to normal tissue and yᵢ=1 corresponds to diseased (e.g., adenoma) tissue; bⱼ are the coefficients to be determined by minimizing the function; xᵢⱼ is the normalized spectral value corresponding to wavelength number j for tissue sample i; and p is the number of wavelength buckets.

Minimizing this function can provide coefficients bⱼ shown in FIG. 6. As can be seen, there can be a significant variation from a value j to a next value j+1. This variation can be due to a characteristic of applying the above least squares regression analysis to spectral data from patients. More specifically, the spectral measurements can be multicollinear—measurements at one wavelength can be highly correlated with nearby wavelengths. Thus, xᵢⱼ can tends to be close to xᵢⱼ₊₁ for a given patient. For such measurements, a regression such as above can tend to result in very erratic coefficients bⱼ. These erratic coefficients can make an analysis of tissue sensitive to missing or inaccurate individual sensor data, as well as to the specific calibration of individual sensors. Such erratic coefficients can also provide a relatively poor predictor of tissue conditions for new patient data.

Smoother values of bj that can be more like those depicted in FIG. 5 or FIG. 7 and that can be more accurate and reliable predictors of outcomes/conditions for new patients can be obtained by minimizing a composite function that can penalize differences between coefficients that have neighboring wavelength numbers (for example, wavelength numbers within one or two of each other). The composite function can include two functions including a first function and a second function. The first function can be a least squares regression function that can be similar to that discussed with respect to FIG. 6. The first function can include a summation of squared differences between known clinical status yᵢ and scores for each sample in the data set. The second function can be a penalty function that can penalize differences between neighboring coefficients bⱼ.

Neighboring coefficients bⱼ can generally be coefficients that can be within a range of one or two wave numbers j of each other. For a given coefficient bⱼ, the “nearest neighbor” coefficients can include bⱼ₋₁ and bⱼ₊₁. The penalty function can penalize differences between neighboring and nearest neighbor coefficients such that variations, such as those shown in FIG. 6, are reduced. That is one characteristic of the method consistent with current subject matter. Another characteristic is that predicted outcomes can be more reliable for new patients. Two examples of composite functions in accordance with some implementations of the current subject matter are discussed below.

A first example of the composite function can include two functions including a first function and a second function. The first function can be a least squares regression function that can utilize training data. This can include known conditions yᵢ and spectral data xᵢⱼ for the known conditions. The first function can be similar to that discussed with respect to FIG. 6.

The second function can penalize differences between pairs of nearest neighbor coefficients. The second function can include a squared sum of the difference between pairs of coefficients bⱼ that are adjacent in j. The sum can be multiplied by constant λ. The constant λ can be optimized via cross-validation or measurement of a the model's fit to a given population of samples. This first example of the function can be as follows:

Σᵢ₌₁ⁿ[yᵢ−b₀−Σⱼ₌₁^{p}bⱼxᵢⱼ]²+λΣⱼ₌₂^{p}(bⱼ−bⱼ₋₁)²

The constant λ in the above sum is a parameter that is used to suppress large variations between pairs of values of bⱼ. The curve in FIG. 5 is an example of a curve generated by minimizing this first example of a modified function.

A second example of the composite function can include two functions including a first function and a second function. The first function can be a least squares regression function that can utilize training data. This can include known conditions yᵢ and spectral data xᵢⱼ for the known conditions. The first function can be similar to that discussed with respect to FIG. 6.

The second function can penalize differences between each coefficient and its nearest neighbors. The second function can include a sum of the squared difference between a coefficient bⱼ and the average of its two nearest neighbors in j. This can penalize coefficients that are substantially different from the average of their nearest neighbors in j. The second sum can be multiplied by constant λ. The constant λ can be optimized via cross-validation or measurement of a the model's fit to a given population of samples. This second example of the function can be as follows:

Σᵢ₌₁ⁿ[yᵢ−b₀−Σⱼ₌₁^{p}bⱼxᵢⱼ]²+λΣⱼ₌₃^{p}(bⱼ−2bⱼ₋₁+bⱼ₋₂)²

The constant λ can be selected to suppress large differences between bⱼ and the average of its neighbors according to j. The curve in FIG. 7 can be an example of a curve generated by minimizing this second example of a modified function.

At least some of the subject matter described herein can be embodied in systems, apparatus, methods, and/or articles depending on the desired configuration. In particular, various implementations of the subject matter described herein can be realized in digital electronic circuitry, integrated circuitry, specially designed application specific integrated circuits (ASICs), computer hardware, firmware, software, and/or combinations thereof. These various implementations can include implementation in one or more computer programs that are executable and/or interpretable on a programmable system including at least one programmable processor, which can be special or general purpose, coupled to receive data and instructions from, and to transmit data and instructions to, a storage system, at least one input device, and at least one output device.

These computer programs, which can also be referred to programs, software, software applications, applications, components, or code, include machine instructions for a programmable processor, and can be implemented in a high-level procedural and/or object-oriented programming language, and/or in assembly/machine language. As used herein, the term “machine-readable medium” refers to any computer program product, apparatus and/or device, such as for example magnetic discs, optical disks, memory, and Programmable Logic Devices (PLDs), used to provide machine instructions and/or data to a programmable processor, including a machine-readable medium that receives machine instructions as a machine-readable signal. The term “machine-readable signal” refers to any signal used to provide machine instructions and/or data to a programmable processor. The machine-readable medium can store such machine instructions non-transitorily, such as for example as would a non-transient solid state memory or a magnetic hard drive or any equivalent storage medium. The machine-readable medium can alternatively or additionally store such machine instructions in a transient manner, such as for example as would a processor cache or other random access memory associated with one or more physical processor cores.

The implementations set forth in the foregoing description do not represent all implementations consistent with the subject matter described herein. Instead, they are merely some examples consistent with aspects related to the described subject matter. Although a few variations have been described in detail above, other modifications or additions are possible. In particular, further features and/or variations can be provided in addition to those set forth herein. For example, the implementations described above can be directed to various combinations and subcombinations of the disclosed features and/or combinations and subcombinations of several further features disclosed above. In addition, the logic flows depicted in the accompanying figures and/or described herein do not necessarily require the particular order shown, or sequential order, to achieve desirable results. Other implementations may be within the scope of the following claims.

What is claimed is:

1. A method comprising:
illuminating a tissue of a body with an excitation wavelength;
receiving, in response to the illumination, light from the tissue;
detecting intensities associated with wavelengths of the received light;
computing, using a plurality of wavelength dependent coefficients determined using a composite function that includes a second function applied to minimize differences between neighboring coefficients, a score that is characterized by a weighted function of the intensities; and
generating, based on the score, an output characterizing a condition of the tissue.

2. The method of claim 1, wherein the composite function comprises a first function and the second function.

3. The method of claim 2, wherein the plurality of wavelength dependent coefficients are determined by minimizing the composite function.

4. The method of claim 1, further comprising:
receiving the plurality of wavelength dependent coefficients.

5. The method of claim 1, wherein the first function is a least squares regression function applied to training data.

6. The method of claim 1, wherein the second function includes a sum of squared differences between neighboring coefficients of the plurality of coefficients.

7. The method of claim 1, wherein an average absolute value of a difference between consecutive coefficients is less than three percent of a range of the plurality of coefficients.

8. The method of claim 5, wherein:
the excitation wavelength is about 337 nanometers; and
the spectral data is associated with a spectral curve disposed between 350 nm and 600 nm.

9. The method of claim 1, wherein the condition of the tissue characterizes whether the tissue is diseased.

10. The method of claim 1, wherein the intensities are detected using a plurality of diodes, each diode being sensitive to a respective band of wavelengths, each detected intensity of the intensities being based on an output of one or more diodes of the plurality of diodes.

11. The method of claim 1, further comprising:
normalizing the intensities such that the intensity values are dimensionless.

12. A system comprising:
at least one programmable processor; and
a non-transitory machine-readable medium storing instructions that, when executed by the at least one processor, cause the at least one programmable processor to perform operations comprising:
receiving data regarding wavelengths of light emitted from a tissue;
determining, based on the wavelengths in the received data and using a composite function comprising a mathematical sum of a first function and a second function, coefficients of spectral analysis data, the second function minimizing differences between neighboring coefficients; and
providing the coefficients, the coefficients being used to generate one or more scores used to generate an output characterizing a condition of the tissue.

13. The system of claim 12, wherein:
the coefficients are determined by minimizing the composite function;
the first function characterizes a least squares regression analysis performed on spectral data associated with a plurality of individuals, the least squares regression analysis being associated with a plurality of initial coefficients; and
the second function characterizes a sum of squared differences between each neighboring coefficients of the plurality of initial coefficients.

14. The system of claim 13, wherein an average absolution value of a difference between consecutive coefficients is less than two percent of a range of the plurality of coefficients.

15. A system comprising:
at least one illumination source configured to illuminate a tissue with an excitation wavelength;
at least one detector configured to perform operations comprising:
receiving, in response to the illumination, light emitted from the tissue; and
detecting intensities corresponding to wavelengths of the emitted light; and
a computational module comprising a non-transitory machine-readable medium storing instructions that, when executed by at least one programmable processor, cause the at least one programmable processor to perform operations comprising:
computing, using a plurality of wavelength dependent coefficients determined by minimizing a sum of a first function and a second function, the second function minimizing a difference between neighboring coefficients, a score that is characterized by a weighted function of the intensities; and
generating, based on the score, an output characterizing a condition of the tissue.

16. The system of claim 15, wherein the computational module further performs operations comprising:
receiving the plurality of wavelength dependent coefficients.

17. The system of claim 15, wherein the first function characterizes a least squares regression analysis performed on spectral data associated with a plurality of individuals.

18. The system of claim 17, wherein the least squares regression analysis is associated with a plurality of coefficients, and wherein the second function includes a sum of terms, each term being proportional to a squared difference between a corresponding coefficient of the plurality of coefficients and an average of at least two coefficients that are nearest neighbors with the corresponding coefficient.

19. The system of claim 17, wherein the least squares regression analysis is associated with a plurality of coefficients, and wherein the second function includes a sum of terms, each term being proportional to a squared difference between two consecutive coefficients.

20. The system of claim 15, wherein:
the excitation wavelength is 337 nanometers; and
the spectral data is associated with a spectral curve disposed between 350 nm and 600 nm.
